(19) United States
(12) Patent Application Publication
(10) Pub. No.: US 2024/0386713 A1
(43) Pub. Date: Nov. 21, 2024

(54) ADDITIVE MULTIPLE INSTANCE LEARNING

(71) Applicant: PathAI, Inc., Boston, MA (US)

(72) Inventors: Syed Ashar Javed, Revere, MA (US); Dinkar Juyal, Boston, MA (US); Harshith Padigela, Boston, MA (US); Amaro N. Taylor-Weiner, Brooklyn, NY (US); Limin Yu, Troy, MI (US); Aaditya Prakash, Newtonville, MA (US)

(73) Assignee: PathAI, Inc., Boston, MA (US)

(21) Appl. No.: 18/660,563

(22) Filed: May 10, 2024

Related U.S. Application Data
(60) Provisional application No. 63/466,434, filed on May 15, 2023.

(51) Int. Cl. G06V 10/82; G06V 10/77

(52) U.S. Cl. CPC G06V 10/82; G06V 10/7715

(57) ABSTRACT

Described herein are methods for performing additive multiple instance learning. A bag comprising patches is generated from an input image using a patch generator. A featurizer having a neural network model is used to generate a plurality of patch embeddings using at least a portion of the bag. An attention module is used to generate an attention score for each of the plurality of patch embeddings. The attention module is further used to generate a plurality of attention weighted patch embeddings by scaling the plurality of patch embeddings using the attention scores. An additive predictor is used to aggregate the plurality of attention weighted patch embeddings to generate a plurality of patch-wise class contributions. Each patch-wise class contribution represents a contribution of a corresponding class. The additive predictor is used to compute a plurality of predictions from the patch-wise class contributions using an additive function.

### CROSS-REFERENCE TO RELATED APPLICATION

This Application claims the benefit under 35 U.S.C. § 119 (e) of U.S. Provisional Application Ser. No. 63/466,434, filed on May 15, 2023, under Attorney Docket No. P1112.70022US00, entitled “ADDITIVE MULTIPLE INSTANCE LEARNING,” which is hereby incorporated herein by reference in its entirety.

### BACKGROUND

Multiple Instance Learning (MIL) has been applied in pathology towards solving critical problems such as automating cancer diagnosis and grading, predicting patient prognosis, and therapy response. Deploying these models in a clinical setting requires careful inspection of these black boxes during development and deployment to identify failures and maintain physician trust.

### SUMMARY

Some embodiments relate to a system for additive multiple instance learning (MIL), comprising at least one processor operatively connected to a memory; a patch generator, executed by the at least one processor, configured to generate a bag comprising a plurality of patches from an input image, each patch comprising a distinct portion of the input image; a featurizer, executed by the at least one processor, comprising a neural network model configured to generate a plurality of patch embeddings using at least a portion of the bag; an attention module, executed by the at least one processor, configured to: determine an attention score for at least some of the plurality of patch embeddings; and generate a plurality of attention weighted patch embeddings by scaling the plurality of patch embeddings using the attention scores; and an additive predictor, executed by the at least one processor, configured to: aggregate the plurality of attention weighted patch embeddings to generate a plurality of patch-wise class contributions, wherein each patch-wise class contribution represents a contribution of a corresponding class; and compute a plurality of predictions from the patch-wise class contributions using an additive function.

In some embodiments, the neural network model is trained with weakly annotated data.

In some embodiments, the additive predictor is further configured to distinguish between excitatory and inhibitory patch contributions using at least one of the plurality of patch-wise class contributions.

In some embodiments, distinguishing between excitatory and inhibitory patch contributions comprises determining the sign of the at least one of the plurality of patch-wise class contributions.

In some embodiments, computing the plurality of predictions comprises computing a first prediction for a first class and a second prediction for a second class.

In some embodiments, the system further comprises a display module configured to display a heatmap of the image, the heatmap identifying patch-wise class contributions associated with the first class and patch-wise class contributions associated with the second class.

In some embodiments, the additive predictor is further configured to perform, using the heatmap, one or more among model debugging, validating model performance, and identifying spurious features.

In some embodiments, using the additive function comprises adding class-wise contribution functions for the plurality of patches together.

In some embodiments, the plurality of patch-wise class contributions are linear.

Some embodiments relate to a method for performing additive multiple instance learning (MIL), comprising generating, using a patch generator, a bag comprising a plurality of patches from an input image, each patch comprising a distinct portion of the input image; generating, using a featurizer comprising a neural network model, a plurality of patch embeddings using at least a portion of the bag; determining, using an attention module, an attention score for at least some of the plurality of patch embeddings; generating, using the attention module, a plurality of attention weighted patch embeddings by scaling the plurality of patch embeddings using the attention scores; aggregating, using an additive predictor, the plurality of attention weighted patch embeddings to generate a plurality of patch-wise class contributions, wherein each patch-wise class contribution represents a contribution of a corresponding class; and computing, using the additive predictor, a plurality of predictions from the patch-wise class contributions using an additive function.

In some embodiments, the neural network model is trained with weakly annotated data.

In some embodiments, the method further comprises distinguishing between excitatory and inhibitory patch contributions using at least one of the plurality of patch-wise class contributions.

In some embodiments, distinguishing between excitatory and inhibitory patch contributions comprises determining the sign of the at least one of the plurality of patch-wise class contributions.

In some embodiments, computing the plurality of predictions comprises computing a first prediction for a first class and a second prediction for a second class.

In some embodiments, the method further comprises displaying a heatmap of the image, the heatmap identifying patch-wise class contributions associated with the first class and patch-wise class contributions associated with the second class.

In some embodiments, the method further comprises performing, using the heatmap, one or more among: model debugging, validating model performance, and identifying spurious features.

In some embodiments, using the additive function comprises adding class-wise contribution functions for the plurality of patches.

In some embodiments, the plurality of patch-wise class contributions are linear.

### BRIEF DESCRIPTION OF THE DRAWINGS

The figures are provided for the purposes of illustration and explanation and are not intended as a definition of the limits of the systems and methods described herein. In the figures:

FIG. 1 is a block diagram illustrating an example of an additive multiple instance learning (MIL) model, in accordance with some embodiments.

FIG. 2 is a table showing how additive MIL models can achieve comparable or superior performance to the standard attention MIL model, in accordance with some embodiments.

FIG. 3A provides a comparison between the precision of an attention MIL model and that of an additive model, in accordance with some embodiments.

FIG. 3B shows a WSI from the Camelyon 16 dataset, in accordance with some embodiments.

FIG. 3C shows a heatmaps generated using an additive MIL, in accordance with some embodiments.

FIG. 3D shows a heatmaps generated using an attention MIL, in accordance with some embodiments.

FIG. 4A shows the sum of patch contribution in a bag for the additive MIL in the case of Kidney Chromophobe (KICH), in accordance with some embodiments.

FIG. 4B shows the sum of patch contribution in a bag for the additive MIL in the case of Kidney renal papillary cell carcinoma (KIRP), in accordance with some embodiments.

FIG. 4C shows the sum of patch contribution in a bag for the additive MIL in the case of Kidney renal clear cell carcinoma (KIRC), in accordance with some embodiments.

FIG. 4D shows median score from top-10% patches in a bag for the attention MIL model in the case of Kidney Chromophobe (KICH), in accordance with some embodiments.

FIG. 4E shows median score from top-10% patches in a bag for the attention MIL model in the case of Kidney renal papillary cell carcinoma (KIRP), in accordance with some embodiments.

FIG. 4F shows median score from top-10% patches in a bag for the attention MIL model in the case of Kidney renal clear cell carcinoma (KIRC), in accordance with some embodiments.

FIG. 5A shows portion of a slide including a renal cell carcinoma (RCC) region, in accordance with some embodiments.

FIG. 5B shows an attention heatmap identifying attention scores associated with FIG. 5A, in accordance with some embodiments.

FIG. 5C shows an additive heatmap identifying KIRC regions and KIRP regions, in accordance with some embodiments.

FIG. 5D shows portion of a slide including a non-small cell lung cancer (NSCLC) region, in accordance with some embodiments.

FIG. 5E shows an attention heatmap identifying attention scores associated with FIG. 5D, in accordance with some embodiments.

FIG. 5F shows an additive heatmap identifying adenocarcinoma regions and squamous cell carcinoma regions, in accordance with some embodiments.

FIG. 6A shows a portion of a slide including a renal cell carcinoma (RCC) region, in accordance with some embodiments.

FIG. 6B shows an additive heatmaps associated with FIG. 6A identifying KIRC regions, in accordance with some embodiments.

FIG. 6C shows an additive heatmaps associated with FIG. 6A identifying KIRP regions, in accordance with some embodiments.

FIG. 6D shows an additive heatmaps associated with FIG. 6A identifying KICH regions, in accordance with some embodiments.

FIG. 7A shows a portion of a slide including an RCC region, in accordance with some embodiments.

FIG. 7B shows an attention heatmap associated with FIG. 7A, in accordance with some embodiments.

FIG. 7C shows an additive heatmap associated with FIG. 7A, in accordance with some embodiments.

FIG. 7D shows a portion of a slide including a Cemalyon 16 dataset, in accordance with some embodiments.

FIG. 7E shows an attention heatmap associated with FIG. 7D, in accordance with some embodiments.

FIG. 7F shows an additive heatmap associated with FIG. 7D, in accordance with some embodiments.

FIG. 8 schematically shows layers of a convolutional neural network. in accordance with some embodiments.

FIG. 9 shows a block diagram of a computer system on which various embodiments may be practiced.

### DETAILED DESCRIPTION

### I. Overview

Described herein is a formulation of Multiple Instance Learning (MIL) models that enables interpretability while maintaining similar predictive performance. The models developed by the inventors and described herein enable spatial credit assignment such that the contribution of each region in an image can be accurately computed and visualized. The resulting spatial credit assignment coincides with regions used by pathologists during diagnosis and improves upon classical attention heatmaps from attention MIL models. These models can debug model failures, identify spurious features, and highlight class-wise regions of interest, enabling their use in high-stakes environments such as clinical decision-making.

Histopathology is the study and diagnosis of disease by microscopic inspection of tissue. Histologic examination of tissue samples plays a key role in both clinical diagnosis and drug development. It is regarded as medicine's ground truth for various diseases and is important in evaluating disease severity, measuring treatment effects, and biomarker scoring. A differentiating feature of digitized tissue slides or whole slide images (WSI) is their extremely large size, often billions of pixels per image. In addition to being large, WSIs are extremely information dense, with each image containing thousands of cells and detailed tissue regions that make manual analysis of these images challenging. This information richness makes pathology an excellent application for machine learning, and indeed there has been tremendous progress in recent years in applying machine learning to pathology data.

One of the most important applications of machine learning in digital pathology involves predicting patient's clinical characteristics from a WSI image. Models need to be able to make predictions about the entire slide involving all the patient tissue available; these predictions are referred to as “slide-level”. To overcome the challenges presented by the large size of these images, previous methods have used smaller hand engineered representations, built from biological primitives in tissue such as cellular composition and structures. Another common way to overcome the challenges presented by the size of WSIs is to break the slide into thousands of small patches, train a model with these patches to predict the slide-label, and then use a secondary model to learn an aggregation function from patch representations to slide-level label. Both methods are not trained in an end-to-end manner and suffer from sub-optimal performance. The second method also suffers from an incorrect assumption that each patch from a slide has the same label as the overall slide.

MIL is a weakly supervised learning technique which attempts to learn a mapping from a set of instances (called a bag) to a single label associated with the whole bag. MIL can be applied to pathology by treating patches from slides as instances which form a bag and a slide-level label is associated with each bag to learn a bag predictor. This circumvents the need to collect patch-level labels and allows end-to-end training from a WSI. The MIL assumption that at least one patch among the set of patches is associated with the target label works well for many biological problems. For example, the MIL assumption holds for the task of cancer diagnosis; a sufficiently large bag of instances or patches from a cancerous slide will contain at least one cancerous patch whereas a benign slide will never contain a cancerous patch. In recent years, attention-based pooling of patches has been shown to be successful for MIL problems. Using neural networks with attention MIL has become the standard for end-to-end pathology models as it provides a powerful, yet efficient gradient based method to learn a slide-to-label mapping. In addition to superior performance, these models encode some level of spatial interpretability within the model through visualization of highly attended regions.

The sensitive nature of the medical imaging domain requires deployed machine learning models to be interpretable for multiple reasons. First, it is critical that models do not learn spurious shortcuts over true signal and can be debugged if such failure modes exist. Interpretability and explainability methods have been shown to help identify some of these data and model deficiencies. Secondly, for algorithms in medical decision-making, accountability and rigorous validation precedes adoption. Interpretable models can be easier to validate and thus build trust. Specifically, users can verify that model predictions are generated using biologically concordant features that are supported by scientific evidence and are similar to the those identified by human experts. Thirdly, use-cases involving a human expert such as decision-support require the algorithm to give a visual cue which highlights the regions to be examined more carefully. In these applications, a predicted score is insufficient and needs to be complemented with a highlighted visual region associated with the model's prediction. For machine learning models in pathology, spatial credit assignment can be defined as attributing model predictions to specific spatial regions in the slide. Various post-hoc interpretability techniques like gradient based methods and Local Interpretable Model-agnostic Explanation (LIME) have been used to this end. However, gradient based methods which try to construct model-dependent saliency maps are often insensitive to the model or the data. This makes these post-hoc methods unreliable for spatial attribution as they provide poor localization and do not reflect the model's predictions.

Model-agnostic methods like Shapley values or LIME involve intractable computations for large image data and thus need approximations like locally fitting explanations to model predictions, which can lead to incorrect attribution. Applying attention MIL in weakly supervised problems in pathology leads to learning of the attention scores for each patch. These scores can be used as a proxy for patch importance, thus helping in spatial credit assignment. This way of interpreting MIL models has been used commonly in the literature to create spatial heatmaps, image overlays that indicate credit assignment, for free without applying any post-hoc technique. The attention values that scale patch feature representations have a non-linear relationship to the final prediction, making their visual interpretation inexact and incomplete.

To address these issues, the inventors propose a formulation of MIL which induces intrinsically interpretable heatmaps. This model is referred to herein as “additive MIL.” It allows for precise decomposition of a model prediction in terms of spatial regions of the input. These models, instead of being applied to arbitrary features, are grounded as patch instances in the MIL formulation which allows precise (e.g., exact) credit assignment for each patch in a bag. Specifically, this is achieved by constraining the space of predictor functions (the classification or regression head at the final layer) in the MIL setup to be additive in terms of instances. Therefore, the contribution of each patch or instance in a bag can be traced back from the final predictions. These additive scores reflect the true marginal contribution of each patch to a prediction and can be visualized as a heatmap on a slide for various applications like model debugging, validating model performance, and identifying spurious features.

The inventors have recognized and appreciated that these benefits can be achieved without any material loss of predictive performance even though the predictor function is constrained to be additive. This represents a substantial improvement over previous MIL implementations.

### II. MIL Models

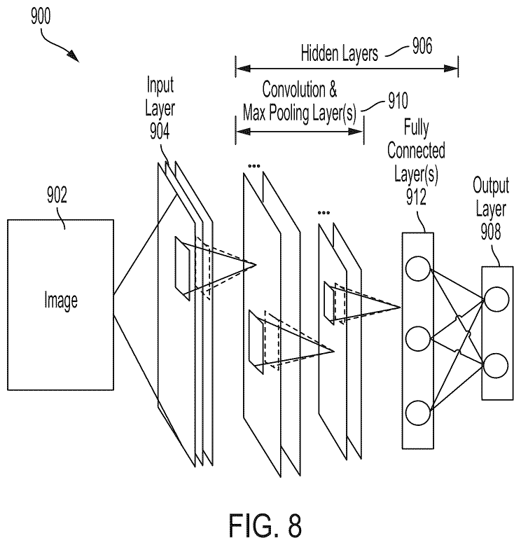

An attention MIL model can be seen as a 3-part model involving (1) a featurizer (f), e.g., a deep convolutional neural network (CNN), an example of which is shown in FIG. 8; (2) an attention module (m), which induces a soft attention over N patches and is used to scale each patch feature; and (3) a predictor (p), which takes the attended patch representations, aggregates them using a permutation invariant function like sum pooling over the N patches, and then outputs a prediction. This MIL model g(x) is given by:

$$g ( x ) = ( p \cdot m \cdot f ) ( x ) \qquad ( 1 )$$

$${m}_{i} ( x ) = {α}_{i} f ( {x}_{i} ) where {α}_{i} = {softmax}_{i} ( {ψ}_{m} ( x ) ) \qquad ( 2 )$$

$$p ( x ) = {ψ}_{p} ( {\sum }_{i = 1}^{N} {m}_{i} ( x ) ) \qquad ( 3 )$$

where ψₘ and ψₚ are multilayer perceptrons (MLPs) with non-linear activation functions. The attention scores αᵢ learned by the model can be treated as patch importance scores and are used to interpret MIL models.

The inventors have recognized and appreciated that there are several limitations in doing spatial attribution using these attention scores. For example, consider the task of classifying a slide into benign, suspicious or malignant.

First, since the attention weights are used to scale the patch features used for the prediction task, a high attention weight only means that the patch might be needed for the prediction downstream. Therefore, a high attention score for a patch can be a necessary but not sufficient condition for attributing a prediction to that patch. Similarly, patches with low attention can be important for the downstream prediction since the attention scores are related non-linearly to the final classification or regression layer. For example, in a malignant slide, non-tumor regions might get highlighted by the attention scores since they need to be represented at the final classification layer to provide discriminative signal. However, this does not imply malignant prediction should be attributed to non-malignant regions, nor that these regions would be useful to guide a human expert.

Second, the contribution of a patch to the final prediction can be either positive (excitatory) or negative (inhibitory), however attention scores do not distinguish between the two. A patch might be providing strong negative evidence for a class but will be highlighted in the same way as a positive patch. For example, benign mimics of cancer are regions which visually look like cancer but are normal benign tissue. These regions are useful for the model to provide negative evidence for the presence of cancer and thus might have high attention scores. While attending to these regions may be useful to the model, they may complicate human interpretation of resulting heatmaps.

Third, attention scores do not provide meaningful information about the class-wise importance of a patch, but only that a patch was weighted by a certain magnitude for generating the prediction. In the case of multiclass classification, this becomes problematic as a high attention score on a patch can mean that it might be useful for any of the multiple classes. Different regions in the slide might be contributing to different classes which are indistinguishable in an attention heatmap. For example, if a patch has high attention weight for benign-suspicious-malignant classification, it can be interpreted as being important for any one or more of the classes. This makes the attention scores ineffective for verifying the role of individual patches for a slide-level prediction.

Fourth, using attention scores to assess patch contribution ignores patch interactions at the classification stage. For example, two different tumor patches might have moderate attention scores, but when taken together in the final classification layer, they might jointly provide strong and sufficient information for the slide being malignant. Thus, computing marginal patch contributions for a bag needs to be done at the classification layer and not the attention layer since attention scores do not capture patch interactions and thus can underestimate or overestimate contributions to the final prediction.

### III. Additive MIL Models

These limitations in interpreting attention MIL heatmaps motivate the formulation of a traceable predictor function, where model predictions can be specified in terms of patch contributions (both positive and negative) for each class. The inventors have developed additive MIL models to address the aforementioned limitations. The inventors have recognized and appreciated that it is desirable that the approaches described herein be intrinsic to the model, as opposed to being post-hoc approaches. This prevents incorrect assumptions about the model without the need for post-hoc modeling. It also prevents many pitfalls of traditional saliency methods.

The inventors have further recognized and appreciated that it is desirable that attribution be performed in terms of instances only. For pathology, this means that the prediction should be attributed to individual patches. This constraint enables expression of bag predictions in terms of marginal instance contributions.

The inventors have further recognized and appreciated that it is desirable that the model be able to distinguish between excitatory and inhibitory patch contributions. Some models provide per-class contributions for classification problems. To enable the desired instance-level credit assignment in MIL, according to some embodiments, the final predictor is re-framed to be an additive function of individual instances. This can be expressed in accordance with the following example expression:

$${p}_{Additive} ( x ) = {\sum }_{i = 1}^{N} {ψ}_{p} ( {m}_{i} ( x ) ) \qquad ( 4 )$$

Making this change results in the final predictor only being able to implement patch-additive functions on top of arbitrarily complex patch representations. This provides both complexity of the learned representations as well as a traceable patch contribution for a given prediction, which solves the spatial credit assignment problem. The function ψₚ (mᵢ(x)) is the class-wise contribution for patch i in the bag. At inference, ψ produces a R^{C}^{ₓ}^{N} for a classification problem where C is the number of classes and N is the number of patches in a bag. Thus, a class-wise score for each patch is obtained, which when summed gives the final logits for the prediction problem. These scores can be visualized by constructing a heatmap from the visual representation of patch-wise contributions for each class. The sign of the patch contribution determines whether the patch is excitatory or inhibitory towards each class since positive values add to the final logit while negative values bring down the final class logit.

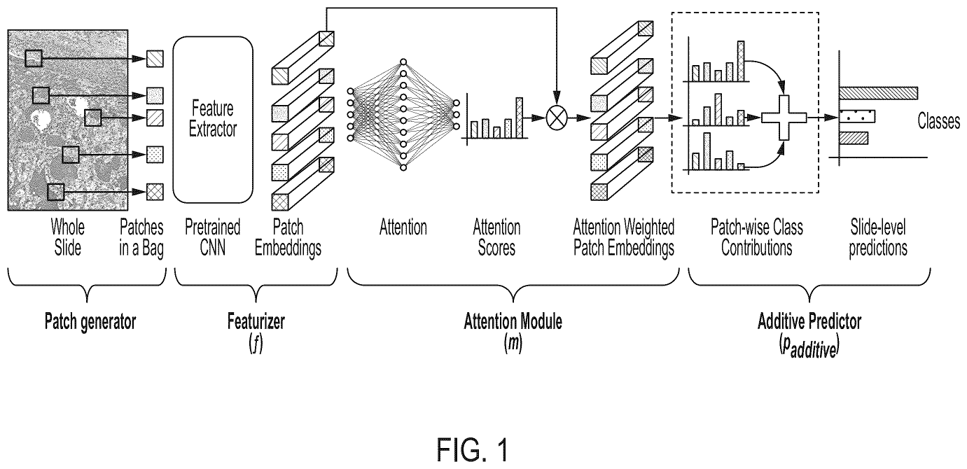

FIG. 1 illustrates an example of an additive MIL model, in accordance with some embodiments. The model includes a patch generator, a featurizer (f), an attention module (m) and an additive predictor (Padditive). The patch generator is configured to generate a bag with a plurality of patches from an input image. Each patch includes a distinct portion of the input image. The featurizer includes a neural network (e.g., convolutional) model configured to generate a plurality of patch embeddings using at least a portion of the bag. The attention module is configured to determine an attention score for each of the plurality of patch embeddings. Further, the attention module generates a plurality of attention weighted patch embeddings by scaling the plurality of patch embeddings using the attention scores. The additive predictor is configured to aggregate the plurality of attention weighted patch embeddings to generate a plurality of patch-wise class contributions. Each patch-wise class contribution represents a contribution of a corresponding class. Further, the additive predictor computes a plurality of predictions from the patch-wise class contributions using an additive function. Optionally, a heatmap of the image may be generated. The heatmap may identifying patch-wise class contributions associated with each class, as described in detail further below.

It should be noted that a convolutional neural network is used as an example of a model that may be used in accordance with some embodiments. However, it should be appreciated that other types of statistical models may alternatively be used, and embodiments are not limited in this respect. Other types of statistical models that may be used include a support vector machine, a neural network, a regression model, a random forest, a clustering model, a Bayesian network, reinforcement learning, metric learning, a genetic algorithm, or another suitable statistical model.

### IV. Aspects of the Additive MIL Model

The first aspect relates to the ability to produce precise marginal patch contribution towards a prediction. Additive MIL models provide precise (e.g., exact) patch contribution scores which are additively related to the prediction. This additive coupling of the model and the interpretability method makes the spatial scores precisely mirror the invariances and the sensitivities of the model, thus making them intrinsically interpretable.

The second aspect relates to class-wise contributions. Additive MIL models allow decomposing the patch contributions and attributing them to individual classes in a classification problem. This allows not only to assign the prediction to a region, but also to determine to which class it contributes. This is helpful in cases where signal for multiple classes exist within the same slide.

The third aspect relates to distinction between excitatory and inhibitory contributions. Additive MIL models allow for both positive and negative contributions from a patch. This can help distinguish between areas which are important because they provide evidence for the prediction and those which provide evidence against.

### V. Experiments and Results

Various experiments were performed to show the benefits of additive MIL models for interpretability in pathology problems. The experiments resulted in one or more of the following effects.

First, additive MIL models provide intrinsic spatial interpretability without material loss of predictive performance as compared to more expressive, non-additive models.

Second, pooling-based MIL model can be made additive by reformulating the predictor function, leading to predictive results similar to the original model.

Third, additive MIL heatmaps yield better alignment with region-annotations from an expert pathologist than attention MIL heatmaps.

Fourth, additive MIL heatmaps provide more granular information like class-wise spatial assignment and excitatory and inhibitory patches which is missing in attention heatmaps. This can be useful for applications like model debugging.

Three different datasets and two different problems were considered in the experiments. The first problem is the prediction of cancer subtypes in non-small cell lung carcinoma (NSCLC) and renal cell carcinoma (RCC), both of which use the TCGA dataset. The second problem is the detection of metastasis in breast cancer using the Camelyon 16 dataset. TCGA RCC contains 966 whole slide images (WSIs) with three histologic subtypes-KICH (chromophobe RCC), KIRC (clear cell RCC) and KIRP (papillary RCC). 768 k patches were extracted from this dataset which translates to an average of 795 patches per slide and 16 k total bags. TCGA NSCLC has 1002 WSIs, with 538 slides belonging to subtype LUAD (Lung Adenocarcinoma) and 464 to LUSC (Lung Squamous Cell Carcinoma). 1.465 million patches were extracted from this dataset which translates to an average of 1462 patches per slide and 30.5 k total bags. Camelyon 16 contains 267 WSIs for training and 129 for testing with a total of 159 malignant slides and 237 benign slides. 510 k patches were extracted from this dataset which translates to an average of 1286 patches per slide and 10.6 k total bags. These numbers point to the diversity in the dataset size in terms of number of slides, number of bags, and the label imbalance.

In some experiments, both TCGA datasets were split into 60/15/25 (train/val/test) while ensuring no data leakage at a case level. For Camelyon 16, the original splits provided with the dataset were used. For training the models, a bag size of 48-1600 patches and batch size of 16-64 was experimented with and the best one chosen using cross-validation. The patches were sampled from non-background regions for all datasets at a resolution of 1 microns per pixel without any overlap between adjacent patches. An ImageNet pre-trained Shufflenet was used as the feature extractor and the entire model was trained with ADAM optimizer and a learning rate of 1e⁻⁴. For inference, multiple bag predictions were aggregated using a majority vote to get the final slide-level prediction. AUROC (area under the receiver operating curve) scores were generated using the proportion of bags predicting the majority label as the class assignment probability. For TCGA-RCC, macro average of 1-vs-rest AUROC was computed across the three classes. The attention scores were obtained by directly taking the raw outputs for each patch from the attention module. For additive patch contributions, the patch-wise class contributions were taken and converted to a bounded patch contribution value using a sigmoid function. This yielded excitatory scores in the range of 0.5-1 and inhibitory scores in the range of 0-0.5. Both the attention and additive patch-wise scores were used for generating a heatmap as an overlay on the slide with attention MIL heatmaps having a single value per patch and additive MIL heatmaps having C values per patch where C is the number of classes. All training and inference runs were done on Quadro RTX 8000, which took three to four hours to train using four GPUs.

### VI. Predictive Performance of Additive MIL Models

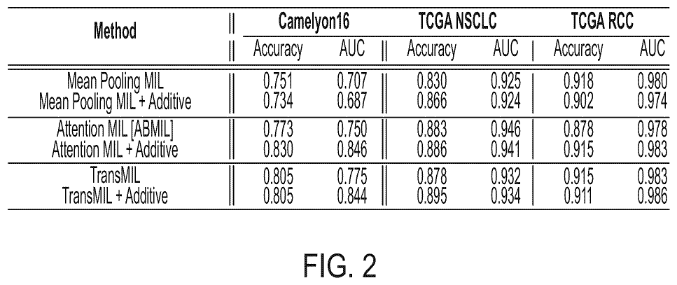

Additive MIL models were compared with existing techniques in terms of predictive performance on three different datasets, as shown in FIG. 2. A mean-pooling based MIL baseline was implemented without any attention, the standard attention MIL model (ABMIL) and a transformer based MIL model, TransMIL which is the state-of-the-art on these three datasets.

In the case of improved performance, it was hypothesized that the additive constraint regularizes the model and limits overfitting in comparison to previous approaches. This is particularly relevant to pathology datasets that often have less than one thousand slides. Implementing the additive formulation gives nearly all the benefits of modeling complexity from previous methods, while enabling spatial interpretability without material loss of predictive performance.

Heatmaps obtained through additive MIL models were compared with heatmaps obtained through attention MIL models. Both were evaluated against region-level annotations from an expert pathologist. The Camelyon 16 dataset was used. The objective was to classify the slide as benign or malignant. Since the cancer foci are very localized and often occupy less than 1% of the slide, the task of generating localized cancer heatmaps in a weakly supervised setup is very challenging. Exhaustive segmentation annotations were obtained for cancer regions from a board-certified pathologist on the Camleyon 16 test set. An additive MIL model was trained accordingly. Traditional attention heatmaps were generated using patch-level attention scores. Additive MIL heatmaps were generated using the patch contributions.

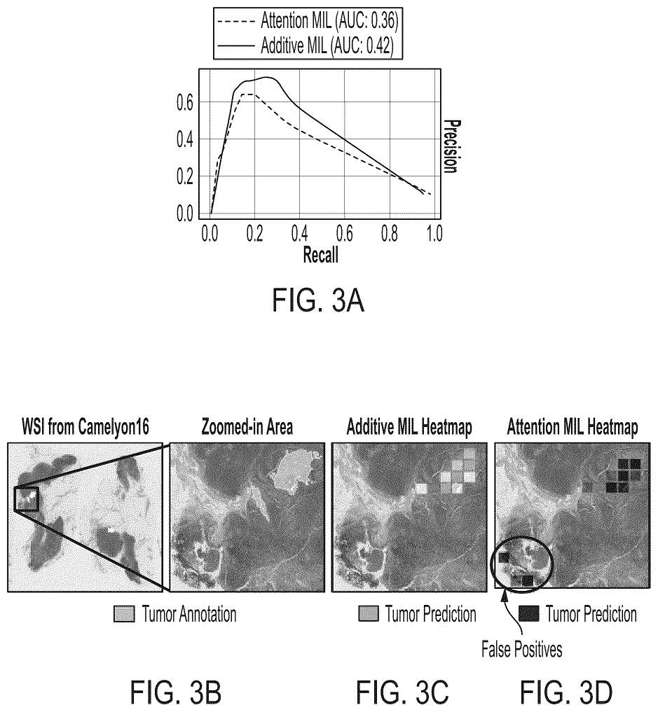

FIG. 3A provides a comparison between the precision of an attention MIL model and that of an additive model, in accordance with one example. More specifically, FIG. 3A shows patch level precision-recall curves at different thresholds of the heatmap. It should be noted that this comparison controls for model performance as both heatmaps are generated from the same model. At low thresholds, nearly all patches are highlighted, and both methods present a high recall and low precision. As the threshold increased, precision is higher and recall is lower.

FIGS. 3B-D provide a comparison between heatmaps generated using an additive MIL and an attention MIL, in accordance with one example. FIG. 3B depicts a WSI from the Camelyon 16 dataset. The additive MIL heatmap shown in FIG. 3C (AUPRC 0.42) highlighted cancer regions more precisely and sensitively than traditional attention heatmaps (AUPRC 0.36), which detect more false-positives, as shown in FIG. 3D. If the best operating point of both of the curves is chosen, the result is that the best FI score for the attention heatmap is 0.43 as compared to 0.47 from the additive heatmap. These experiments demonstrate the superior performance of additive MIL heatmaps in localizing areas of interest, at least in some circumstances.

### VII. Faithful Representation of Patch-level Contributions to Slide-level Predictions

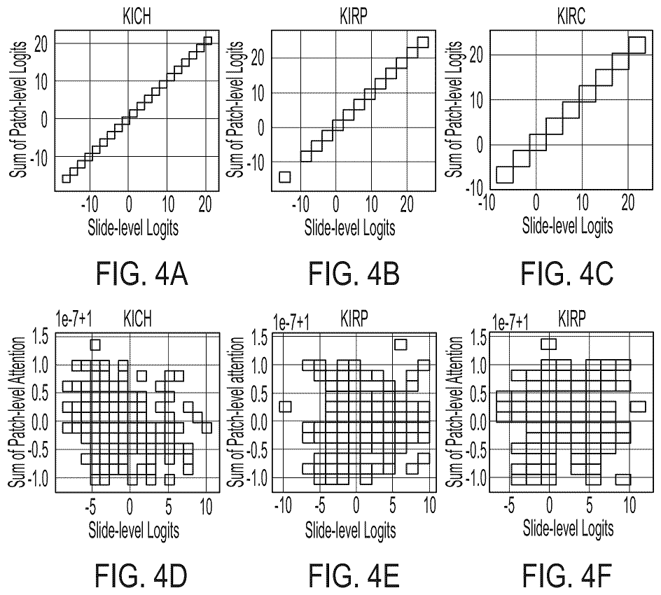

Attention heatmaps are often used to signal regions of interest in a slide. However, as explained above, it is not straightforward to draw conclusions regarding the importance and contribution of attended areas towards the model prediction. Additive MIL guarantees that each patch's contribution is linear and thus faithfully represents its marginal contribution toward the slide-level prediction. This property is shown in FIGS. 4A-4F, illustrating the alignment between the slide-level predicted logits and patch contributions from the additive and the attention models on TCGA RCC. In the top row, the Y-axis shows the sum of patch contribution in a bag for the additive MIL in the case of Kidney Chromophobe (KICH) (FIG. 4A), Kidney renal papillary cell carcinoma (KIRP) (FIG. 4B) and Kidney renal clear cell carcinoma (KIRC) (FIG. 4C). In the bottom-row, the Y-axis shows the median score from top-10% patches in a bag for the attention MIL model in the case of KICH (FIG. 4D), KIRP (FIG. 4E) and KIRC (FIG. 4F). The columns represent the slide-level logits for each class. The colors represent the ground-truth. As can be appreciated, additive contributions are linear, while attention contributions are not (there is no clear relationship with the final predictions).

### VIII. Qualitative Assessment of Multi-Class & Excitatory-Inhibitory Heatmaps

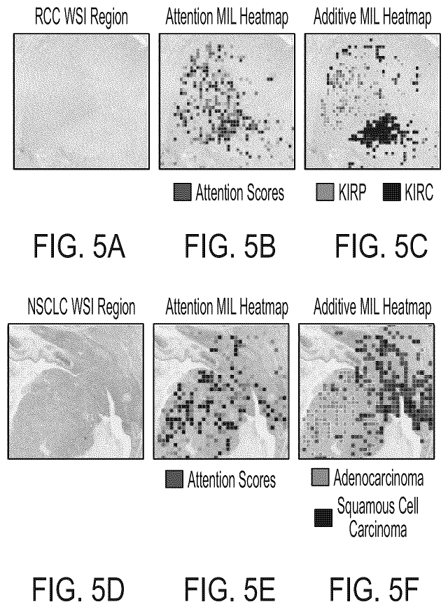

We highlight the benefits of having class-wise excitatory-inhibitory contributions for each spatial region in a slide. FIGS. 5A-5C show a renal cell carcinoma (RCC) region (FIG. 5A), an attention heatmap identifying attention scores (FIG. 5B) and an additive heatmap identifying KIRC regions and KIRP regions (FIG. 5C). FIGS. 5D-5F show a non-small cell lung cancer (NSCLC) region (FIG. 5D), an attention heatmap identifying attention scores (FIG. 5E) and an additive heatmap identifying adenocarcinoma regions and squamous cell carcinoma regions (FIG. 5F). In these examples, the attention heatmaps (heatmaps obtained using an attention MIL model) highlight tissue regions predictive of the cancer subtype, but do not provide information about the association of patches to classes. In contrast, the additive heatmaps (heatmaps obtained using an additive MIL model) show precisely how each patch contributes to each class, and in turn the final prediction. Thus, unlike attention heatmaps, additive heatmaps can visualize class-level information.

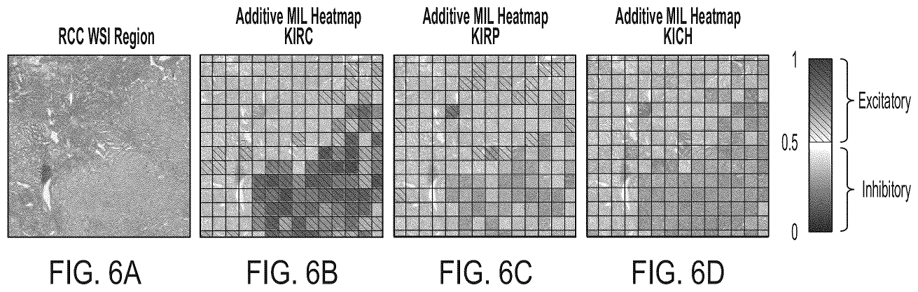

Further, additive MIL models are able to distinguish between excitatory and inhibitory patch contributions. FIG. 6A shows a renal cell carcinoma (RCC) region, and FIGS. 6B-6D show related additive heatmaps identifying KIRC regions (FIG. 6B), KIRP regions (FIG. 6C) and KIRH regions (FIG. 6D). The additive MIL heatmaps for each class are visualized by the same colorbar where red denotes excitatory patches and blue denotes inhibitory ones. The RCC WSI is labeled as KIRC, but the selected region contains two subtypes, namely KIRC and small regions of KIRP, as evident from the raw slide. The additive MIL heatmaps accurately show bottom right region being excitatory for KIRC, but inhibitory for the other two whereas the top left region is only excitatory for KIRP and inhibitory for two other two. All patches are correctly inhibitory for KICH. Such granularity in heatmaps is helpful in understanding how the model arrives at a prediction and can prove to be useful for practitioners building the models as well as physicians using them.

### IX. Model Debugging Using Additive MIL Heatmaps

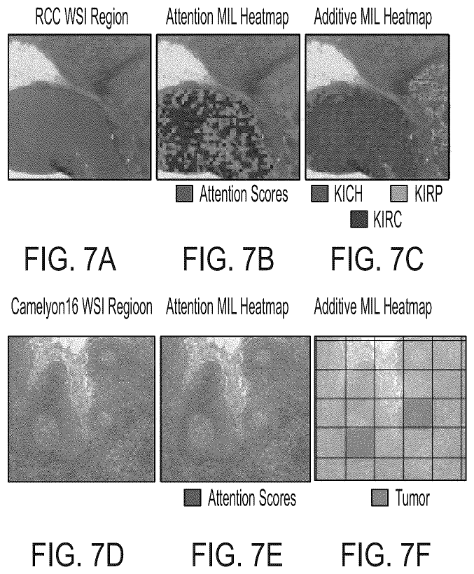

The ability of additive MIL models to accurately reflect model predictions at a patch-level can be useful in model debugging. FIG. 7A-7C show an example of a model mis-predicting a KIRP slide as KICH. FIG. 7A shows a portion of a slide including an RCC region. The attention heatmap of FIG. 7B shows only a region of adrenal gland on the left being attended. However, the additive MIL heatmap of FIG. 7C is able to exactly show how adrenal glands being rare, are being confused for KICH regions even though the model correctly identifies the KIRP regions on the right side.

FIG. 7B shows an example of a Camelyon 16 case where the model is mis-predicting a benign slide as malignant. The attention heatmap (FIG. 7B) offers no useful information. However, the additive MIL heatmap (FIG. 7C) highlights areas of germinal center as the source of this false positive prediction. This pattern for false positive prediction is found in multiple other slides and can enable to go from interpretation to debugging.

These heatmaps not only provide interpretability to MIL models, but can also aid in validating specific hypothesis during model debugging.

### X. Exemplary Neural Network

FIG. 8 schematically shows layers of a convolutional neural network in accordance with some embodiments of the technology described herein. Convolutional neural network 900 may be used to output predictions for a pathology image in accordance with some embodiments of the technology described herein. For example, convolutional neural network 900 may be used to output predictions for a pathology image. The convolutional neural network may be used because such networks are suitable for analyzing visual images. The convolutional neural network may require no pre-processing of a visual image in order to analyze the visual image.

As shown, the convolutional neural network comprises an input layer 904 configured to receive information about the image 902 (e.g., pixel values for all or one or more portions of a pathology image), an output layer 908 configured to provide the output (e.g., a classification), and a plurality of hidden layers 906 connected between the input layer 904 and the output layer 908. The plurality of hidden layers 906 include convolution and pooling layers 910 and fully connected layers 912.

The input layer 904 may be followed by one or more convolution and pooling layers 910. A convolutional layer may comprise a set of filters that are spatially smaller (e.g., have a smaller width and/or height) than the input to the convolutional layer (e.g., the image 902). Each of the filters may be convolved with the input to the convolutional layer to produce an activation map (e.g., a 2-dimensional activation map) indicative of the responses of that filter at every spatial position. The convolutional layer may be followed by a pooling layer that down-samples the output of a convolutional layer to reduce its dimensions. The pooling layer may use any of a variety of pooling techniques such as max pooling and/or global average pooling. In some embodiments, the down-sampling may be performed by the convolution layer itself (e.g., without a pooling layer) using striding.

The convolution and pooling layers 910 may be followed by fully connected layers 912. The fully connected layers 912 may comprise one or more layers each with one or more neurons that receives an input from a previous layer (e.g., a convolutional or pooling layer) and provides an output to a subsequent layer (e.g., the output layer 908). The fully connected layers 912 may be described as “dense” because each of the neurons in a given layer may receive an input from each neuron in a previous layer and provide an output to each neuron in a subsequent layer. The fully connected layers 912 may be followed by an output layer 908 that provides the output of the convolutional neural network. The output may be, for example, an indication of which class, from a set of classes, the image 902 (or any portion of the image 902) belongs to. The convolutional neural network may be trained using a stochastic gradient descent type algorithm or another suitable algorithm. The convolutional neural network may continue to be trained until the accuracy on a validation set (e.g., held out images from the training data) saturates or using any other suitable criterion or criteria.

It should be appreciated that the convolutional neural network shown in FIG. 8 is only one example implementation and that other implementations may be employed. For example, one or more layers may be added to or removed from the convolutional neural network shown in FIG. 8. Additional example layers that may be added to the convolutional neural network include: a pad layer, a concatenate layer, and an upscale layer. An upscale layer may be configured to upsample the input to the layer. An ReLU layer may be configured to apply a rectifier (sometimes referred to as a ramp function) as a transfer function to the input. A pad layer may be configured to change the size of the input to the layer by padding one or more dimensions of the input. A concatenate layer may be configured to combine multiple inputs (e.g., combine inputs from multiple layers) into a single output.

As another example, in some embodiments, one or more convolutional, transpose convolutional, pooling, unpooling layers, and/or batch normalization may be included. As yet another example, the architecture may include one or more layers to perform a nonlinear transformation between pairs of adjacent layers. The non-linear transformation may be a rectified linear unit (ReLU) transformation, a sigmoid, and/or any other suitable type of non-linear transformation, as aspects of the technology described herein are not limited in this respect. In some embodiments, any suitable optimization technique may be used for estimating neural network parameters from training data. For example, one or more of the following optimization techniques may be used: stochastic gradient descent (SGD), mini-batch gradient descent, momentum SGD, Nesterov accelerated gradient, Adagrad, Adadelta, RMSprop, Adaptive Moment Estimation (Adam), AdaMax, Nesterov-accelerated Adaptive Moment Estimation (Nadam), AMSGrad.

Convolutional neural networks may be employed to perform any of a variety of functions described herein. It should be appreciated that more than one convolutional neural network may be employed to make predictions in some embodiments. For example, a first convolutional neural network may be trained on a set of annotated pathology images and a second, different convolutional neural network may be trained on the same set of annotated pathology images, but magnified by a particular factor, such as 5×, 10×, 20×, or another suitable factor. The first and second neural networks may comprise a different arrangement of layers and/or be trained using different training data. In some embodiments, the convolutional neural network does not include padding between layers. The layers may be designed such that there is no overflow as pooling or convolution operations are performed. Moreover, layers may be designed to be aligned. For example, if a layer has an input of size N*N, and has a convolution filter of size K, with stride S, then (N-K)/S must be an integer in order to have alignment.

### XI Exemplary Computer Architecture

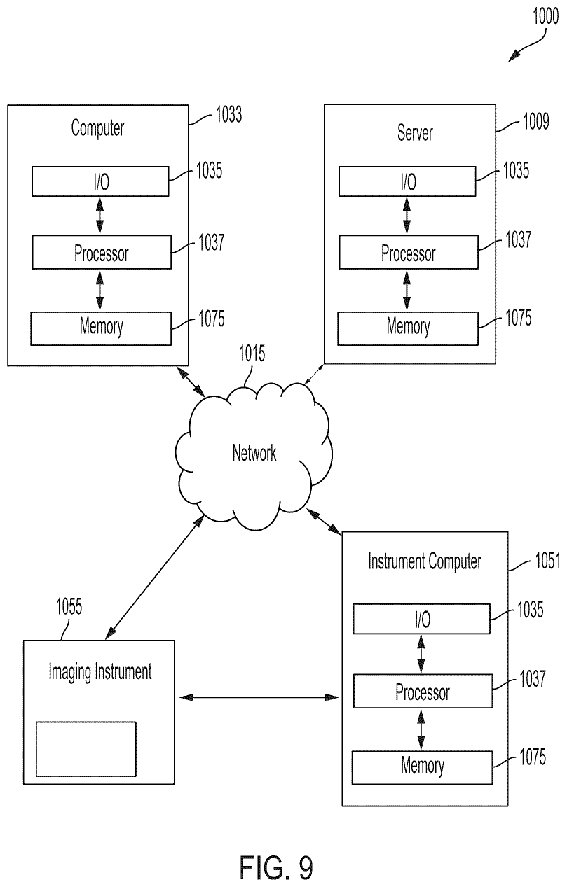

FIG. 9 shows a block diagram of a computer system on which various embodiments of the technology described herein may be practiced. The system 1000 includes at least one computer 1033. Optionally, the system 1000 may further include one or more of a server computer 1009 and an imaging instrument 1055 (e.g., one of the instruments described above), which may be coupled to an instrument computer 1051. Each computer in the system 1000 includes a processor 1037 coupled to a tangible, non-transitory memory device 1075 and at least one input/output device 1035. Thus, the system 1000 includes at least one processor 1037 coupled to a memory subsystem 1075 (e.g., a memory device or collection of memory devices). The components (e.g., computer, server, instrument computer, and imaging instrument) may be in communication over a network 1015 that may be wired or wireless and wherein the components may be remotely located or located in close proximity to each other. Using those components, the system 1000 is operable to receive or obtain image data such as pathology images, histology images, or tissue images and annotation and score data as well as test sample images generated by the imaging instrument or otherwise obtained. In certain embodiments, the system uses the memory to store the received data as well as the model data which may be trained and otherwise operated by the processor.

In some embodiments, some or all of system 1000 is implemented in a cloud-based architecture. The cloud-based architecture may offer on-demand access to a shared pool of configurable computing resources (e.g., processors, graphics processors, memory, disk storage, network bandwidth, and other suitable resources). A processor in the cloud-based architecture may be operable to receive or obtain training data such as pathology images, histology images, or tissue images and annotation and score data as well as test sample images generated by the imaging instrument or otherwise obtained. A memory in the cloud-based architecture may store the received data as well as the model data which may be trained and otherwise operated by the processor. In some embodiments, the cloud-based architecture may provide a graphics processor for training the model in a faster and more efficient manner compared to a conventional processor.

Processor refers to any device or system of devices that performs processing operations. A processor will generally include a chip, such as a single core or multi-core chip (e.g., 12 cores), to provide a central processing unit (CPU). In certain embodiments, a processor may be a graphics processing unit (GPU) such as an Nvidia Tesla K80 graphics card from NVIDIA Corporation (Santa Clara, CA). A processor may be provided by a chip from Intel or AMD. A processor may be any suitable processor such as the microprocessor sold under the trademark XEON E5-2620 v3 by Intel (Santa Clara, CA) or the microprocessor sold under the trademark OPTERON 6200 by AMD (Sunnyvale, CA). Computer systems may include multiple processors including CPUs and or GPUs that may perform different steps of the described methods. The memory subsystem 1075 may contain one or any combination of memory devices. A memory device is a mechanical device that stores data or instructions in a machine-readable format.

Memory may include one or more sets of instructions (e.g., software) which, when executed by one or more of the processors of the disclosed computers can accomplish some or all of the methods or functions described herein. Each computer may include a non-transitory memory device such as a solid-state drive, flash drive, disk drive, hard drive, subscriber identity module (SIM) card, secure digital card (SD card), micro-SD card, or solid-state drive (SSD), optical and magnetic media, others, or a combination thereof. Using the described components, the system 1000 is operable to produce a report and provide the report to a user via an input/output device. An input/output device is a mechanism or system for transferring data into or out of a computer. Exemplary input/output devices include a video display unit (e.g., a liquid crystal display (LCD) or a cathode ray tube (CRT)), a printer, an alphanumeric input device (e.g., a keyboard), a cursor control device (e.g., a mouse), a disk drive unit, a speaker, a touchscreen, an accelerometer, a microphone, a cellular radio frequency antenna, and a network interface device, which can be, for example, a network interface card (NIC), Wi-Fi card, or cellular modem.

### XII. Conclusion

Described herein is a novel type of MIL model for use in pathology (and other applications) that makes models intrinsically interpretable through an additive function. The approach described herein enables exact spatial credit assignment where the final prediction of the model can be attributed to individual contributions of each patch in a pathology slide. These models provide spatial interpretability without material loss of predictive performance and can be used for various applications like model debugging and highlighting regions-of-interest in a decision-support setting. This high fidelity interpretability can be critical in building trust for these models when deployed in medical decision-making.

It is to be appreciated that embodiments of the methods and apparatuses discussed herein are not limited in application to the details of construction and the arrangement of components set forth in the present disclosure or illustrated in the accompanying drawings. The methods and apparatuses are capable of implementation in other embodiments and of being practiced or of being carried out in various ways. Examples of specific implementations are provided herein for illustrative purposes only and are not intended to be limiting. In particular, any embodiment disclosed herein may be combined with any other embodiment in any manner consistent with at least one of the objects, aims, and needs disclosed herein, and references to “an embodiment,” “some embodiments,” “an alternate embodiment,” “various embodiments,” “one embodiment” or the like are not necessarily mutually exclusive and are intended to indicate that a particular feature, structure, or characteristic described in connection with the embodiment may be included in at least one embodiment. The appearances of such terms herein are not necessarily all referring to the same embodiment.

Also, the phraseology and terminology used herein is for the purpose of description and should not be regarded as limiting. Any references to embodiments or elements or acts of the systems and methods herein referred to in the singular may also embrace embodiments including a plurality of these elements, and any references in plural to any embodiment or element or act herein may also embrace embodiments including only a single element. References in the singular or plural form are not intended to limit the presently disclosed systems or methods, their components, acts, or elements.

Also, various inventive concepts may be embodied as one or more processes, of which examples have been provided. The acts performed as part of each process may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, or ordinary meanings of the defined terms.

The use herein of “including,” “comprising,” “having,” “containing,” “involving,” and variations thereof is meant to encompass the items listed thereafter and equivalents thereof as well as additional items. References to “or” may be construed as inclusive so that any terms described using “or” may indicate any of a single, more than one, and all of the described terms. Any references to front and back, left and right, top and bottom, upper and lower, and vertical and horizontal are intended for convenience of description, not to limit the present systems and methods or their components to any one positional or spatial orientation.

As referred to herein, the term “in response to” may refer to initiated as a result of or caused by. In a first example, a first action being performed in response to a second action may include interstitial steps between the first action and the second action. In a second example, a first action being performed in response to a second action may not include interstitial steps between the first action and the second action.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In this application, unless otherwise clear from context, (i) the term “a” means “one or more”; (ii) the term “or” is used to mean “and/or” unless explicitly indicated to refer to alternatives only or the alternative are mutually exclusive, although the disclosure supports a definition that refers to only alternatives and “and/or”; (iii) the terms “comprising” and “including” are understood to encompass itemized components or steps whether presented by themselves or together with one or more additional components or steps; and (iv) where ranges are provided, endpoints are included.

Use of ordinal terms such as “first,” “second,” “third,” etc., in the claims to modify a claim element does not by itself connote any priority, precedence, or order of one claim element over another or the temporal order in which acts of a method are performed. Such terms are used merely as labels to distinguish one claim element having a certain name from another element having the same name (but for use of the ordinal term).

Having thus described several aspects of at least one embodiment, it is to be appreciated that various alterations, modifications, and improvements will readily occur to those skilled in the art. Such alterations, modifications, and improvements are intended to be part of this disclosure and are intended to be within the spirit and scope of the systems and methods described herein. Accordingly, the foregoing description and drawings are by way of example only.

What is claimed is:

1. A system for additive multiple instance learning (MIL), comprising:
at least one processor operatively connected to a memory;
a patch generator, executed by the at least one processor, configured to generate a bag comprising a plurality of patches from an input image, each patch comprising a distinct portion of the input image;
a featurizer, executed by the at least one processor, comprising a neural network model configured to generate a plurality of patch embeddings using at least a portion of the bag;
an attention module, executed by the at least one processor, configured to:
determine an attention score for at least some of the plurality of patch embeddings; and
generate a plurality of attention weighted patch embeddings by scaling the plurality of patch embeddings using the attention scores; and
an additive predictor, executed by the at least one processor, configured to:
aggregate the plurality of attention weighted patch embeddings to generate a plurality of patch-wise class contributions, wherein each patch-wise class contribution represents a contribution of a corresponding class; and
compute a plurality of predictions from the patch-wise class contributions using an additive function.

2. The system of claim 1, wherein the neural network model is trained with weakly annotated data.

3. The system of claim 1, wherein the additive predictor is further configured to distinguish between excitatory and inhibitory patch contributions using at least one of the plurality of patch-wise class contributions.

4. The system of claim 3, wherein distinguishing between excitatory and inhibitory patch contributions comprises determining the sign of the at least one of the plurality of patch-wise class contributions.

5. The system of claim 1, wherein computing the plurality of predictions comprises computing a first prediction for a first class and a second prediction for a second class.

6. The system of claim 5, further comprising a display module configured to display a heatmap of the image, the heatmap identifying patch-wise class contributions associated with the first class and patch-wise class contributions associated with the second class.

7. The system of claim 6, wherein the additive predictor is further configured to perform, using the heatmap, one or more among:
model debugging,
validating model performance, and
identifying spurious features.

8. The system of claim 1, wherein using the additive function comprises adding class-wise contribution functions for the plurality of patches together.

9. The system of claim 1, wherein the plurality of patch-wise class contributions are linear.

10. A method for performing additive multiple instance learning (MIL), comprising:
generating, using a patch generator, a bag comprising a plurality of patches from an input image, each patch comprising a distinct portion of the input image;
generating, using a featurizer comprising a neural network model, a plurality of patch embeddings using at least a portion of the bag;
determining, using an attention module, an attention score for at least some of the plurality of patch embeddings;
generating, using the attention module, a plurality of attention weighted patch embeddings by scaling the plurality of patch embeddings using the attention scores;
aggregating, using an additive predictor, the plurality of attention weighted patch embeddings to generate a plurality of patch-wise class contributions, wherein each patch-wise class contribution represents a contribution of a corresponding class; and
computing, using the additive predictor, a plurality of predictions from the patch-wise class contributions using an additive function.

11. The method of claim 10, wherein the neural network model is trained with weakly annotated data.

12. The method of claim 10, further comprising distinguishing between excitatory and inhibitory patch contributions using at least one of the plurality of patch-wise class contributions.

13. The method of claim 12, wherein distinguishing between excitatory and inhibitory patch contributions comprises determining the sign of the at least one of the plurality of patch-wise class contributions.

14. The method of claim 10, wherein computing the plurality of predictions comprises computing a first prediction for a first class and a second prediction for a second class.

15. The method of claim 14, further comprising displaying a heatmap of the image, the heatmap identifying patch-wise class contributions associated with the first class and patch-wise class contributions associated with the second class.

16. The method of claim 15, further comprising performing, using the heatmap, one or more among:
model debugging,
validating model performance, and
identifying spurious features.

17. The method of claim 10, wherein using the additive function comprises adding class-wise contribution functions for the plurality of patches.

18. The method of claim 10. wherein the plurality of patch-wise class contributions are linear.
